(19) United States
(12) Patent Application Publication
(10) Pub. No.: US 2023/0172857 A1
(43) Pub. Date: Jun. 8, 2023

(54) NOVEL RECONSTITUTED HIGH DENSITY LIPOPROTEIN NANOPARTICLE

(71) Applicant: Mepsgenus Inc., Atlanta, GA (US)

(72) Inventors: Hoon Suk RHO, Seoul (KR); Hyuck KIM, Seoul (KR)

(21) Appl. No.: 18/062,507

(22) Filed: Dec. 6, 2022

(30) Foreign Application Priority Data
Dec. 6, 2021 (KR) 10-2021-017330

(51) Int. Cl. A61K 9/127; B82Y 5/00; A61P 25/28

(52) U.S. Cl. CPC A61K 9/1275; A61P 25/28; B82Y 5/00

(57) ABSTRACT

The present disclosure relates to a reconstituted high density lipoprotein (rHDL) nanoparticle and a composition for preventing or treating a neurodegenerative disease comprising the same. Specifically, the present disclosure relates to a rHDL nanoparticle prepared by mixing a phospholipid and an apolipoprotein, and the rHDL nanoparticle of the present disclosure has amyloid-beta (Aβ) aggregation inhibitory effect.

### CROSS-REFERENCE TO RELATED APPLICATIONS

This application claims the right of priority to Republic of Korea application no. KR 10- 2021-0173303, filed Dec. 6, 2021, which is herein incorporated in its entirety by reference.

### TECHNICAL FIELD

The present disclosure relates to a reconstituted high density lipoprotein (rHDL) nanoparticle and a composition for preventing or treating a neurodegenerative disease comprising the same. Specifically, the present disclosure relates to a rHDL nanoparticle comprising a phospholipid and apolipoprotein E as an active ingredient, and the rHDL nanoparticle of the present disclosure has excellent amyloid-beta (Aβ) aggregation inhibitory effect.

### BACKGROUND

Recently, along with the rapid increase of the elderly population, interest in treatment and prevention of various neurodegenerative diseases is increasing due to the increase in patients with various neurodegenerative diseases. Neurodegenerative disease is a disease that causes various pathologies such as movement disorders, memory disorders, and cognitive disorders due to the decrease or loss of nerve cell function. Nerve cells die in large numbers every day not only in nervous system diseases but also in normal adult brains, and the number of nerve cells that die increases exponentially with aging.

Major diseases belonging to neurodegenerative diseases include Alzheimer’s disease, Parkinson’s disease, Lou Gehrig’s disease, Huntington’s disease, and the like, and the pathogenesis of the disease has not been fully elucidated until now. Acetylcholinesterase inhibitors, NMDA (N-methyl-D-aspartate) receptor antagonists, and the like are used as therapeutic agents for Alzheimer’s disease, and L-dopa, dopamine agonists, MAO-B inhibitors, COMT inhibitors, and the like are used as therapeutic agents for Parkinson’s disease, and dopamine D2 receptors and the like are used as therapeutic agents for Huntington’s disease. However, the above therapeutic agents are known to only delay the onset or alleviate symptoms rather than a fundamental treatment. Accordingly, there is a continuous demand for novel drugs capable of fundamental prevention or treatment.

In particular, Alzheimer’s disease (AD) is the most common form of dementia and is a representative neurodegenerative disease. It is estimated that more than 20% of the elderly over 80 years of age are affected by Alzheimer’s disease, and the number is rapidly increasing as the aging society increases. The main pathological features of Alzheimer’s disease are a senile plaque in which Amyloid-beta (Aβ), which is produced by sequential cleavage of amyloid precursor protein (APP) by β and γ-secretase, is deposited in brain tissue, and neurofibrillary tangle (NTF) caused by hyperphosphorylation of Tau protein, a microtubule-associated protein. In particular, accumulation of Aβ, a type of protein, in the brain damages the blood-brain barrier (BBB) and slows the delivery of essential nutrients from the brain blood vessels to the nerve cells, as well as it causes continuous inflammation of nerve cells, interfering with the activity of immune cells such as microglial cells, and eventually neutralizes the role of synapses, the area where nerve cells send and receive signals. It is known that a series of pathophysiological factors eventually lead to a gradual decline in human intellectual and daily life functions such as memory, judgment, and language ability, and to cause personality behavioral disorders. Therefore, suppressing excessive Aβ production in the brain, or preventing aggregation of the produced Aβ, or effectively reducing the aggregated Aβ structure is considered an important part in the prevention and treatment of Alzheimer’s disease.

On the other hand, high density lipoprotein (HDL) is a major cholesterol carrier in the body formed based on an apolipoprotein, and is characterized by high density (>1.063 g/ml) and small size (stoke diameter = 5 to 17 nm). Mature HDL particles are present in the form of spheres containing cholesterol, phospholipids and various apolipoproteins, and the like. Polar lipids, phospholipids and free cholesterol are present in the outer layer of these particles, and more hydrophobic lipids such as esterified cholesterol and triglycerides are present in the center of the particles. Newly formed or nascent HDL particles in the liver and intestines lack lipids and are present in the form of discoids. The protein component is contained in the outer layer, and the main protein component, apolipoprotein, includes apolipoprotein A1 (Apo A1), which is known as the main protein in the blood, and it is known that it includes, depending on its function, Apo A2, Apo A4, Apo C3, Apo D, Apo E, Apo J and Apo M, and the like.

Apo A1 is synthesized in the liver and intestine and controls the physiological action of HDL in the blood, and plays a role in removing cholesterol from surrounding tissues and transporting it back to the liver or other lipoproteins by the reverse cholesterol transport (RCT) mechanism. It has been known that due to the physiological function of HDL in the blood based on Apo A1, it can prevent and treat many risk factors that cause arteriosclerosis, and therefore, research on a reconstituted high density lipoprotein has been conducted based on Apo A1 for decades.

In contrast, it is known that Apo E is synthesized in the liver and neuroglia and is responsible for regulating the homeostasis of cholesterol and lipids in the brain. In the case of humans, it is known that Apo E2, Apo E3, and Apo E4 are present as types of isoform proteins, and a combination of these affects homeostasis of the aging brain and thus the incidence of Alzheimer’s disease varies. It is reported that Apo E4 mainly causes Alzheimer’s disease, which is known to be involved in amyloid-beta (Aβ) aggregation and eventually damage the BBB. On the other hand, it is reported that Apo E2 and Apo E3 play a protective role. It is known that Apo E2, Apo E3, and Apo E4 are formed by mutation of two amino acids, and the lipidation pattern and receptor binding affinity are changed due to these gene mutations. The LDLR (low density lipoprotein receptor) receptor group, to which Apo E is mainly bound, is widely distributed in the BBB and may have a significant effect on Apo E’s entry and exit into the brain.

It is known that patients with cognitive disorder or Alzheimer’s disease have significantly lower HDL levels than normal people. Accordingly, there have been attempts to prevent cognitive disorder including Alzheimer’s disease using HDL. However, HDL dynamically reacts with various apolipoproteins in the body and coexists with each other, and the diversity of HDL isolated from human plasma has made it difficult to study the analytical mechanism.

### DESCRIPTION OF INVENTION

### Technical Problem

An object of the present invention is to provide rHDL that has therapeutic effects and does not exhibit toxicity in vivo.

Another object of the present invention is to provide a composition for preventing or treating a neurodegenerative disease, comprising rHDL.

Another object of the present invention is to provide a method for treating a neurodegenerative disease, comprising administering rHDL to a patient with a neurodegenerative disease.

### Solution to Problem

The present disclosure provides a reconstituted high density lipoprotein (rHDL) comprising a phospholipid; and at least one of apolipoprotein E (e.g., Apo E2 and/or Apo E3).

The present disclosure provides a reconstituted high density lipoprotein, wherein the reconstituted high density lipoprotein comprises a phospholipid; and apolipoprotein E, and has been generated from a mixture comprising the phospholipid, and apolipoprotein E at the blending weight ratio of 0.25:1 to 2.5:1, and preferably 0.5:1 to 1.5:1.

In one embodiment, the weight ratio of the phospholipid, and apolipoprotein E in the reconstituted high density lipoprotein (rHDL) is 0.2:1 to 2.5:1, preferably 0.2:1 to 1.5:1, and more preferably 0.2:1 to 0.5:1.

In one embodiment, the density of the reconstituted high density lipoprotein (rHDL) is 0.1 to 2.0 g/ml, preferably 0.2 to 1.5 g/ml, and more preferably 0.3 to 1.2 g/ml.

In one embodiment, the apolipoprotein E is apolipoprotein E2, E3, or E2 and E3

In one embodiment, the apolipoprotein further comprises apolipoprotein A1.

The present disclosure provides a composition for preventing or treating a neurodegenerative disease, comprising the reconstituted high density lipoprotein. In one embodiment, the reconstituted high density lipoprotein can inhibit aggregation of amyloid-beta (Aβ) and maintain brain tissue homeostasis.

The present disclosure provides a method for treating a neurodegenerative disease, comprising administering the reconstituted high density lipoprotein to a patient with a neurodegenerative disease.

The present disclosure provides a method for preparing a reconstituted high density lipoprotein (rHDL) comprising a phospholipid; and at least one of apolipoprotein E, comprising the steps of: injecting a phospholipid solution into a second inlet located in the middle of a microfluidic device comprising three inlets and one outlet, and injecting an apolipoprotein solution into a first inlet and a third inlet located on both sides. In one embodiment, the microfluidic device may comprise a micropillar.

In one embodiment, the synthesis blending weight ratio of the phospholipid, and apolipoprotein E is 0.25:1 to 2.5:1, and preferably 0.5:1 to 1.5:1.

The present disclosure provides a reconstituted high density lipoprotein (rHDL) prepared by the preparation method.

### Effects of Invention

The rHDL nanoparticle of the present disclosure and a composition comprising rHDL nanoparticle having an effect of inhibiting aggregation of amyloid-beta (Aβ). Therefore, the rHDL nanoparticle of the present disclosure can be used for the prevention or treatment of a neurodegenerative disease.

In addition, since the rHDL of the present disclosure can be prepared using a microfluidic device, mass production of rHDL with consistent characteristics (no batch-to-batch variability) is possible through continuous synthesis through a single-step process. It does not exhibit toxicity in vivo because it does not contain additional other ingredients such as surfactants compared to conventional rHDL.

### BRIEF DESCRIPTION OF DRAWINGS

FIG. 1 is a schematic diagram of a microfluidic device for preparing rHDL nanoparticles. The microfluidic device utilizes the effect that apolipoproteins dissolved in a hydrophilic solution and phospholipids dissolved in a lipophilic solution are strongly mixed as they go through the continuously propagating microvortex caused by micropillars.

FIGS. 2 and 3 are graphs showing the homogeneity of the production yield and particle size depending on the presence or absence of micropillars in the microfluidic device for producing the rHDL nanoparticles shown in FIG. 1, and show the effect of continuously propagating microvortex on the formation of nanoparticles.

FIGS. 4 and 5 illustrate the size and distribution of nanoparticles depending on the synthesis blending ratio of phospholipid (DMPC) and apolipoprotein E3, and the production amount (volume) depending on each ratio.

FIGS. 6 and 7 illustrate the size and distribution of nanoparticles depending on the synthesis blending ratio of phospholipid (DPPC) and apolipoprotein E3, and the production amount (volume) depending on each ratio.

FIGS. 8 and 9 illustrate the size and distribution of nanoparticles depending on the synthesis blending ratio of phospholipid (POPC) and apolipoprotein E3, and the production amount (volume) depending on each ratio.

FIG. 10 illustrates a DLS measurement result for comparing the size distribution of apolipoproteins E2 and E3 and the size distribution of rHDL nanoparticles formed using them. Apolipoproteins E2 and E3 do not have a difference in size, and the rHDL nanoparticles formed through this conjugate with phospholipids to form large particles of about 5 to 10 nm, and these also do not have a difference caused by apolipoproteins.

FIG. 11 is a TEM photograph of rHDL nanoparticles containing apolipoprotein E3 prepared in a synthesis blending weight ratio of phospholipid and apolipoprotein of 0.75:1.

FIG. 12 is a TEM photograph of rHDL nanoparticles containing apolipoprotein E3 prepared in a synthesis blending weight ratio of phospholipid and apolipoprotein of 1.25: 1.

FIG. 13 is a TEM photograph of rHDL nanoparticles containing apolipoprotein E3 prepared in a synthesis blending weight ratio of phospholipid and apolipoprotein of 2.5:1.

FIG. 14 illustrates a DLS measurement result of confirming a uniform distribution when hybrid rHDL nanoparticles containing apolipoproteins E2 and E3 simultaneously are prepared.

FIG. 15 illustrates the results showing that phospholipids can be contained relatively little and more depending on the structural analysis of the molecular form of rHDL nanoparticles containing apolipoprotein E3.

FIG. 16 illustrates the results for the size of rHDL nanoparticles containing apolipoprotein E3 after synthesis for a case in which phospholipids are contained relatively little and more

FIG. 17 is a graph showing the Aβ aggregation inhibitory effect of rHDL nanoparticles containing apolipoprotein E2 at each concentration over time.

FIG. 18 is a graph showing the Aβ aggregation inhibitory effect of rHDL nanoparticles containing apolipoprotein E3 at each concentration over time.

FIG. 19 is a graph showing the Aβ aggregation inhibitory effect of hybrid rHDL nanoparticles containing apolipoproteins E2 and E3 simultaneously at each concentration over time.

FIG. 20 is a graph showing the Aβ aggregation inhibitory effect of rHDL nanoparticles depending on the type of apolipoprotein contained in the rHDL nanoparticles at each concentration over time.

FIG. 21 is a graph comparing the Aβ aggregation inhibitory effect between pure apolipoprotein E3 and rHDL nanoparticles formed using the same over time.

FIG. 22 is a graph showing the results of an experiment on the amount of uptake in cells for rHDL nanoparticles containing apolipoprotein E2 or E3, and hybrid rHDL nanoparticles containing apolipoproteins E2 and E3 in order to show transport (transcytosis) into brain tissue mediated by brain microvascular endothelial cells (BMEC).

FIG. 23 is a graph showing the results of an experiment on the amount of uptake in cells for rHDL nanoparticles containing apolipoprotein E2 or E3, and hybrid rHDL nanoparticles containing apolipoproteins E2 and E3 in order to show transport mediated by astrocytes of the blood-brain barrier.

FIG. 24 illustrates the results of photographing the brain tissue distribution of amyloid after administration of the saline solution to an animal model of Alzheimer’s disease (5xFAD) for 3 months.

FIG. 25 illustrates the results of photographing the brain tissue distribution of amyloid after administration of the rHDL nanoparticles containing apolipoprotein E3 to an animal model of Alzheimer’s disease (5xFAD) for 3 months.

FIG. 26 illustrates the results showing the concentration of amyloid in CSF after administration of the rHDL nanoparticles containing apolipoprotein E3 to a normal mouse (wild type; WT) and an animal model of Alzheimer’s disease (5xFAD) for 3 months.

FIG. 27 illustrates the results showing the concentration of amyloid in the plasma after administration of the rHDL nanoparticles containing apolipoprotein E3 to a normal mouse (wild type; WT) and an animal model of Alzheimer’s disease (5xFAD) for 3 months.

### DETAILED DESCRIPTION OF THE INVENTION

Hereinafter, with reference to the accompanying drawings, embodiments and examples of the present disclosure will be described in detail so that those of ordinary skill in the art to which the present disclosure belongs can easily carry out. However, the present disclosure can be embodied in various forms and is not limited to the embodiments and examples described herein.

Throughout the present specification, when a certain part “includes” a certain component, it means that other components can be further included, rather than excluding other components, unless otherwise stated.

The term “apolipoprotein E” refers to a mammalian protein encoded by the APOE gene or a functional variant thereof. In preferred embodiments, the apolipoprotein E is a human protein encoded by the human APOE gene on chromosome 19. The apolipoprotein E can be any one of the isoforms of the APOE gene product, such as apolipoprotein E2 (“APOE2”), apolipoprotein E3 (“APOE3”) and apolipoprotein E4 (APOE4). APOE is polymorphic with three major alleles (epsilon 2, epsilon 3, and epsilon 4). Any of the alleles can be used in various embodiments of the present invention. Its “functional variant” refers to a variant of the mammalian protein encoded by the APOE gene maintaining the same or similar biological function as the APOE gene product. In some cases, the functional variant includes amino acid insertion, deletion, and/or substitution compared to the protein encoded by the human APOE gene. In some cases, the functional variant is a fragment of the protein encoded by the human APOE gene.

In some embodiments, apolipoprotein E2 is a protein, having a sequence disclosed in GenBank with the accession no. ARQ79459 or at least 95% sequence identity thereto, preferably at least 98% or 99% sequence identity. In some embodiments, apolipoprotein E3 is a protein having a sequence disclosed in GenBank with the accession no. ARQ79461.1 or at least 95% sequence identity thereto, preferably at least 98% or 99% sequence identity.

In some embodiments, apolipoprotein E in the reconstituted high density lipoprotein (rHDL) is a recombinant protein generated by genetic engineering, or a synthetic protein generated by chemical synhesis.

The term “apolipoprotein A1” refers to a mammalian protein encoded by the APOA1 gene or a functional variant thereof. In preferred embodiments, the apolipoprotein A1 is a human protein encoded by the human APOA1 gene located on chromosome 11. Its “functional variant” refers to a variant of the mammalian protein encoded by the APOA1 gene maintaining the same or similar biological function as the APOA1 gene product. In some cases, the functional variant includes amino acid insertion, deletion, and/or substitution compared to the protein encoded by the human APOA1 gene. In some cases, the functional variant is a fragment of the protein encoded by the human APOA1 gene.

In some embodiments, apolipoprotein A1 is a protein having a sequence disclosed in GenBank with the accession no. AAS68227.1 or at least 95% sequence identity thereto, preferably at least 98% or 99% sequence identity.

In some embodiments, apolipoprotein A1 in the reconstituted high density lipoprotein (rHDL) is a recombinant protein generated by genetic engineering, or a synthetic protein generated by chemical synhesis.

The present disclosure provides a reconstituted high density lipoprotein (rHDL) comprising a phospholipid; and at least one of apolipoprotein E.

As used herein, the term “reconstituted high density lipoprotein (rHDL)” refers to a non-naturally occurring HDL-like particle comprising a phospholipid and apolipoprotein.

In one embodiment, apolipoprotein E is apolipoprotein E2, or E3. In another embodiment, the apolipoprotein includes apolipoproteins E2 and E3 simultaneously, and may further include apolipoprotein A1. In some embodiments, the reconstituted high density lipoprotein (rHDL) comprises apolipoprotein E2 or E3, and A1. In some embodiments, the reconstituted high density lipoprotein (rHDL) comprises apolipoprotein E2, E3 and A1.

In some embodiments, the reconstituted high density lipoprotein (rHDL) comprises apolipoprotein E2 and/or E3 and no other apolipoprotein. In some embodiments, the reconstituted high density lipoprotein (rHDL) comprises apolipoprotein A1, E2 and/or E3 and no other apolipoprotein. In some embodiments, the reconstituted high density lipoprotein (rHDL) is free of apolipoprotein E4. In some embodiments, the reconstituted high density lipoprotein (rHDL) is free of esterified cholesterol or triglycerides. In some embodiments, the reconstituted high density lipoprotein (rHDL) is free of apolipoprotein A1, A2, A4, C3, D, J or M.

As used herein, the term “phospholipid” may be at least one selected from the group consisting of, for example, 1,2-dioleoyl-sn-glycero-3-phosphatidylcholine (DOPC), egg phosphatidylcholine (EPC), dilauroylphosphatidylcholine (DLPC), 1,2-dimyristoyl-sn-glycero-3-phosphocholine (DMPC), dipalmitoylphosphatidylcholine (DPPC), distearoylphosphatidylcholine (DSPC), 1-myristoyl-2-palmitoylphosphatidylcholine (MPPC), 1-palmitoyl-2-myristoylphosphatidylcholine (PMPC), 1-palmitoyl-2-stearoylphosphatidylcholine (PSPC), 1-stearoyl-2-palmitoyl phosphatidylcholine (SPPC), 1,2-distearoyl-sn-glycero-3-phosphocholine (DAPC), 1,2-diarachidoyl-sn-glycero-3-phosphocholine (DBPC), 1,2-dieicosanoyl-sn-glycero-3-phosphocholine (DEPC), palmitoyloleoylphosphatidylcholine (POPC), lysophosphatidylcholine, dilinoleoylphosphatidylcholine, distearoylphosphatidylethanolamine (DSPE), dimyristoylphosphatidylethanolamine (DMPE), dipalmitoylphosphatidylethanolamine (DPPE), palmitoyloleoylphosphatidylethanolamine (POPE), lysophosphatidylethanolamine, N1-[2-((1S)-1-[(3-aminopropyl)amino]-4-[di(3-aminopropyl)amino]butylcarboxamido)ethyl]-3,4-di[oleyloxy]-benzamide) (VL-5), dioctadecylamidoglycylspermine 4-trifluoroacetic acid (DOGS), 3β-[N-(N′,N′-dimethylaminoethane)-carbamoyl]cholesterol (DC-Chol), 1,2-di-O-octadecenyl-3-trimethylammonium propane (DOTMA), 1,2-dioleyl-3-trimethylammonium-propane (DOTAP), (1,2-dioleyloxypropyl)-3-dimethylhydroxyethyl ammonium bromide (DORIE), 1,2-dimyristyloxy-propyl-3-dimethyl-hydroxy ethyl ammonium bromide (DMRIE), 2,3-dioleyloxy-N-[2-(sperminecarboxamido)ethyl]-N,N-dimethyl-1-propanaminium trifluoroacetate (DOSPA), N-(3-aminopropyl)-N,N-dimethyl-2,3-bis(dodecyloxy)-1-propanammonium bromide (GAP-DLRIE), N-t-butyl-N′-tetradecyl-3-tetradecylaminopropionamidine (diC14-amidine), ethylphosphocholine (ethyl PC), dimethyldioctadecylammonium bromide (DDAB), N4-cholesteryl-spermine (GL67), 1,2-dioleyloxy-3-dimethylaminopropane (DODMA), or D-Lin-MC3-DMA (MC3, DLin-MC3-DMA), DLin-KC2-DMA, DLin-DMA, but is not limited thereto.

In some embodiments, a reconstituted high density lipoprotein comprises one, two, three or four phospholipids selected from 1,2-dioleoyl-sn-glycero-3-phosphatidylcholine (DOPC), egg phosphatidylcholine (EPC), dilauroylphosphatidylcholine (DLPC), 1,2-dimyristoyl-sn-glycero-3-phosphocholine (DMPC), dipalmitoylphosphatidylcholine (DPPC), distearoylphosphatidylcholine (DSPC), 1-myristoyl-2-palmitoylphosphatidylcholine (MPPC), 1-palmitoyl-2-myristoylphosphatidylcholine (PMPC), 1-palmitoyl-2-stearoylphosphatidylcholine (PSPC), 1-stearoyl-2-palmitoyl phosphatidylcholine (SPPC), 1,2-distearoyl-sn-glycero-3-phosphocholine (DAPC), 1,2-diarachidoyl-sn-glycero-3-phosphocholine (DBPC), 1,2-dieicosanoyl-sn-glycero-3-phosphocholine (DEPC), palmitoyloleoylphosphatidylcholine (POPC), lysophosphatidylcholine, dilinoleoylphosphatidylcholine, distearoylphosphatidylethanolamine (DSPE), dimyristoylphosphatidylethanolamine (DMPE), dipalmitoylphosphatidylethanolamine (DPPE), palmitoyloleoylphosphatidylethanolamine (POPE), lysophosphatidylethanolamine, N1-[2-((1S)-1-[(3-aminopropyl)amino]-4-[di(3-aminopropyl)amino]butylcarboxamido)ethyl]-3,4-di[oleyloxy]-benzamide) (VL-5), dioctadecylamidoglycylspermine 4-trifluoroacetic acid (DOGS), 3β-[N-(N′,N′-dimethylaminoethane)-carbamoyl]cholesterol (DC-Chol), 1,2-di-O-octadecenyl-3-trimethylammonium propane (DOTMA), 1,2-dioleyl-3-trimethylammonium-propane (DOTAP), (1,2-dioleyloxypropyl)-3-dimethylhydroxyethyl ammonium bromide (DORIE), 1,2-dimyristyloxy-propyl-3-dimethyl-hydroxy ethyl ammonium bromide (DMRIE), 2,3-dioleyloxy-N-[2-(sperminecarboxamido)ethyl]-N,N-dimethyl-1-propanaminium trifluoroacetate (DOSPA), N-(3-aminopropyl)-N,N-dimethyl-2,3-bis(dodecyloxy)-1-propanammonium bromide (GAP-DLRIE), N-t-butyl-N′-tetradecyl-3-tetradecylaminopropionamidine (diC14-amidine), ethylphosphocholine (ethyl PC), dimethyldioctadecylammonium bromide (DDAB), N4-cholesteryl-spermine (GL67), 1,2-dioleyloxy-3-dimethylaminopropane (DODMA), or D-Lin-MC3-DMA (MC3, DLin-MC3-DMA), DLin-KC2-DMA, and DLin-DMA.

In some embodiments, a reconstituted high density lipoprotein comprises DMPC. In some embodiments, a reconstituted high density lipoprotein comprises DPPC. In some embodiments, a reconstituted high density lipoprotein comprises POPC.

In some embodiments, a reconstituted high density lipoprotein comprises DMPC and no other phospholipid. In some embodiments, a reconstituted high density lipoprotein comprises DPPC and no other phospholipid. In some embodiments, a reconstituted high density lipoprotein comprises POPC and no other phospholipid.

The present disclosure provides a reconstituted high density lipoprotein, wherein the reconstituted high density lipoprotein comprises a phospholipid; and apolipoprotein E. In some embodiments, the synthesis blending for the reconstituted high density lipoprotein has the phospholipid, and apolipoprotein E at a weight ratio of 0.25:1 to 2.5:1, and preferably 0.5:1 to 1.5:1. In one embodiment, the apolipoprotein E is apolipoprotein E2, E3, or E2 and E3.

In some embodiments, the reconstituted high density lipoprotein (rHDL) has been generated from a mixture comprising the phospholipid, and apolipoprotein E at a weight ratio of 0.25:1 to 2.5:1. In some embodiments, the reconstituted high density lipoprotein (rHDL) has been generated from a mixture comprising the phospholipid, and apolipoprotein E at a weight ratio of 0.5:1 to 1.5:1. In some embodiments, the reconstituted high density lipoprotein (rHDL) has been generated from a mixture comprising the phospholipid, and apolipoprotein E at a weight ratio of 0.1:1 to 5:1.

The present disclosure provides a reconstituted high density lipoprotein comprising a phospholipid; and at least one of apolipoprotein E. In some embodiments, the weight ratio of the phospholipid, and apolipoprotein E in the reconstituted high density lipoprotein (rHDL) is 0.1: 1 to 5:1. In some embodiments, the weight ratio of the phospholipid, and apolipoprotein E in the reconstituted high density lipoprotein (rHDL) is 0.1:1 to 3:1. In some embodiments, the weight ratio of the phospholipid, and apolipoprotein E in the reconstituted high density lipoprotein (rHDL) is 0.2:1 to 1.5:1. In some embodiments, the weight ratio of the phospholipid, and apolipoprotein E in the reconstituted high density lipoprotein (rHDL) is 0.2: 1 to 0.5:1.

In some embodiments, the weight ratio of the phospholipid, apolipoprotein E2 and apolipoprotein E3 in the reconstituted high density lipoprotein (rHDL) is 1:5:5 to 30:5:5. In some embodiments, the weight ratio of the phospholipid, apolipoprotein E2 and apolipoprotein E3 in the reconstituted high density lipoprotein (rHDL) is 2:5:5 to 15:5:5. In some embodiments, the weight ratio of the phospholipid, apolipoprotein E2 and apolipoprotein E3 in the reconstituted high density lipoprotein (rHDL) is 2:5:5 to 5:5:5.

In some embodiments, the weight ratio of apolipoprotein E2 and apolipoprotein E3 in the reconstituted high density lipoprotein (rHDL) is 1:10 to 10:1. In some embodiments, the weight ratio of apolipoprotein E2 and apolipoprotein E3 in the reconstituted high density lipoprotein (rHDL) is 1:5 to 5:1. In some embodiments, the weight ratio of apolipoprotein E2 and apolipoprotein E3 in the reconstituted high density lipoprotein (rHDL) is 1:2 to 2:1. In some embodiments, the weight ratio of apolipoprotein E2 and apolipoprotein E3 in the reconstituted high density lipoprotein (rHDL) is 1:1.

In some embodiments, the density of the reconstituted high density lipoprotein (rHDL) is 0.1 to 2.0 g/ml. In some embodiments, the density of the reconstituted high density lipoprotein (rHDL) is 0.2 to 1.5 g/ml. In some embodiments, the density of the reconstituted high density lipoprotein (rHDL) is 0.3 to 1.2 g/ml.

In some embodiments, the reconstituted high density lipoprotein (rHDL) is a nanoparticle having a long axis length less than 50 nm, less than 25 nm, or less than 20 nm. In some embodiments, the reconstituted high density lipoprotein (rHDL) is a nanoparticle having a long axis length greater than 1 nm, 2 nm, 3 nm, 4 nm, 5 nm, 6 nm, 7 nm, 8 nm, 9 nm, or 10 nm. In some embodiments, the reconstituted high density lipoprotein (rHDL) is a nanoparticle having a long axis length of 1 to 100 nm, 5 to 50 nm, 5 to 25 nm, or 5 to 20 nm.

In some embodiments, the reconstituted high density lipoprotein (rHDL) is a nanoparticle of 5 to 200 kDa, 5 to 150 kDa, 5 to 100 kDa, or 5 to 60 kDa.

In one aspect, the present disclosure provides a pharmaceutical composition comprising the reconstituted high density lipoprotein described herein and a pharmaceutically acceptable excipient.

In some embodiments, the pharmaceutical composition comprises a first set of the reconstituted high density lipoprotein comprising apolipoprotein E2 and a second set of the reconstituted high density lipoprotein comprising apolipoprotein E3. In some embodiments, the pharmaceutical composition comprises a reconstituted high density lipoprotein comprising both apolipoprotein E2 and apolipoprotein E3.

In some embodiments, the pharmaceutical composition comprises a first set of the reconstituted high density lipoprotein comprising apolipoprotein E2, a second set of the reconstituted high density lipoprotein comprising apolipoprotein E3, and a third set of reconstituted high density lipoprotein comprising apolipoprotein A1. In some embodiments, the pharmaceutical composition comprises a reconstituted high density lipoprotein comprising apolipoprotein E2, E3 and A 1.

In some embodiments, at least 80% of the reconstituted high density lipoproteins (rHDLs) in the pharmaceutical composition are nanoparticles having a long axis length between 10 nm and 20 nm. In some embodiments, at least 70% of the reconstituted high density lipoproteins (rHDLs) in the pharmaceutical composition are nanoparticles having a long axis length between 10 nm and 20 nm, In some embodiments, at least 60% of the reconstituted high density lipoproteins (rHDLs) in the pharmaceutical composition are nanoparticles having a long axis length between 10 nm and 20 nm, In some embodiments, at least 50% of the reconstituted high density lipoproteins (rHDLs) in the pharmaceutical composition are nanoparticles having a long axis length between 10 nm and 20 nm, In some embodiments, at least 40% of the reconstituted high density lipoproteins (rHDLs) in the pharmaceutical composition are nanoparticles having a long axis length between 10 nm and 20 nm,

In some embodiments, at least 80% of the reconstituted high density lipoproteins (rHDLs) in the pharmaceutical composition are nanoparticles having a long axis length between 5 nm and 50 nm. In some embodiments, at least 70% of the reconstituted high density lipoproteins (rHDLs) in the pharmaceutical composition are nanoparticles having a long axis length between 5 nm and 50 nm, In some embodiments, at least 60% of the reconstituted high density lipoproteins (rHDLs) in the pharmaceutical composition are nanoparticles having a long axis length between 5 nm and 50 nm, In some embodiments, at least 50% of the reconstituted high density lipoproteins (rHDLs) in the pharmaceutical composition are nanoparticles having a long axis length between 5 nm and 50 nm, In some embodiments, at least 40% of the reconstituted high density lipoproteins (rHDLs) in the pharmaceutical composition are nanoparticles having a long axis length between 5 nm and 50 nm.

In another aspect, the present disclosure provides a composition for preventing or treating a neurodegenerative disease, comprising the reconstituted high density lipoprotein. In one embodiment, the reconstituted high density lipoprotein can inhibit aggregation of amyloid-beta (Aβ). In some embodiments, the reconstituted high density lipoprotein can maintain brain tissue homeostasis.

As used herein, the term “prevention” refers to any action of inhibiting or delaying the onset of a disease by administration of a composition, and “treatment” refers to any action in which symptoms of a subject suspected of and suffering from a disease are improved or beneficially changed by administration of a composition.

In one embodiment, the neurodegenerative disease is Parkinson’s disease, Alzheimer’s disease, Pick’s disease, Huntington’s disease, Creutzfeldt-Jakob disease, Lou Gehrig’s disease, spinal cerebellar degeneration, spinal cerebellar ataxia, prion disease, cognitive dysfunction, senile dementia, Lewy body dementia, frontotemporal dementia, vascular dementia, alcoholic dementia, presenile dementia, Machado-Joseph disease, myodystonia, multiple system atrophy, progressive supranuclear palsy, Friedreich ataxia, temporal lobe epilepsy, or stroke, but is not limited thereto.

The present disclosure provides a method for preparing a reconstituted high density lipoprotein (rHDL) comprising a phospholipid; and at least one of apolipoprotein E. The method can comprise the steps of: injecting a hydrophilic solution comprising apolipoprotein E into a first inlet of a microfluidic device, injecting a phospholipid solution into a second inlet of the microfluidic device, and collecting the reconstituted high density lipoprotein (rHDL) from an outlet of the microfluidic device.

The present disclosure also provides a method for preparing a reconstituted high density lipoprotein (rHDL) comprising a phospholipid; and at least one of apolipoprotein, comprising the steps of: injecting a first hydrophilic solution comprising a first apolipoprotein into a first inlet of a microfluidic device comprising three inlets and one outlet, injecting a phospholipid solution into a second inlet of the microfluidic device, and injecting a second hydrophilic solution comprising a second apolipoprotein into a third inlet of the microfluidic device.

In some embodiments, the microfluidc device comprises the second inlet in the middle and the first and the third inlet on one of two sides of the microfluidic device. In some embodiments, the microfluidc device further comprises a micropillar configured to mix the phospholipid solution and the hydrophilic solution.

In some embodiments, both the first apolipoprotein and the second apolipoprotein are apolipoprotein E2. In some embodiments, both the first apolipoprotein and the second apolipoprotein are apolipoprotein E3. In some embodiments, the first apolipoprotein is apolipoprotein E2 and the second apolipoprotein is apolipoprotein E3. In some embodiment, the first apolipoprotein is apolipoprotein E2 or E3 and the second apolipoprotein is apolipoprotein A1.

In some embodiments, the first hydrophilic solution, the phospholipid solution, and the second hydrophilic solution are injected concurrently.

In some embodiments, the method further comprises collecting the reconstituted high density lipoprotein (rHDL) from the outlet.

In some embodiments, the weight ratio of the phospholipid, and the first and the second apolipoprotein injected into the inlets of the microfluidic device is 0.25:1 to 2.5:1. In some embodiments, the weight ratio of the phospholipid, and the first and the second apolipoprotein injected into the inlets of the microfluidic device is 0.5:1 to 1.5:1. In some embodiments, the weight ratio of the phospholipid, and the first and the second apolipoprotein injected into the inlets of the microfluidic device is 0.1: 1 to 5:1.

In some embodiments, the weight ratio of the phospholipid, the first apolipoprotein and the second apolipoprotein injected into the inlets of the microfluidic device is 1:2:2 to 10:2:2. In some embodiments, the weight ratio of the phospholipid, and the first apolipoprotein and the second apolipoprotein injected into the inlets of the microfluidic device is 1:1:1 to 3:1:1. In some embodiments, the weight ratio of the phospholipid, the first apolipoprotein and the second apolipoprotein injected into the inlets of the microfluidic device is 1:5:5 to 30:5:5. In some embodiments, the weight ratio of the phospholipid, the first apolipoprotein and the second apolipoprotein injected into the inlets of the microfluidic device is 2:5:5 to 15:5:5. In some embodiments, the weight ratio of the phospholipid, the first apolipoprotein and the second apolipoprotein injected into the inlets of the microfluidic device is 2:5:5 to 5:5:5.

In some embodiments, the method comprises the steps of: injecting a phospholipid solution into a second inlet located in the middle of a microfluidic device comprising three inlets and one outlet, and injecting an apolipoprotein solution into a first inlet and a third inlet located on both sides. In one embodiment, the microfluidic device may comprise a micropillar.

As used herein, the term “microfluidic device” refers to a device including a microchannel provided to allow a fluid to flow on a substrate made of various materials including plastic, glass, metal, or silicon including organic polymer materials, etc.

As used herein, the term “micropillar” or “micropillar structure” refers to a pillar-shaped structure for efficiently mixing different types of fluids injected into the microfluidic device by forming a vortex within the microfluidic device.

In some embodiments, the method further comprises the step of purifying the reconstituted high density lipoprotein (rHDL) by centrifugation.

In one aspect, the present disclosure provides a reconstituted high density lipoprotein (rHDL) prepared by the preparation method described herein.

In another aspect, the present disclosure provides a method for preventing or treating a neurodegenerative disease in a subject, comprising administerating to the subject the reconstituted high density lipoprotein described herein or the pharmaceutical composition described herein. In some embodiments, the disease is selected from the group consisting of Parkinson’s disease, Alzheimer’s disease, Pick’s disease, Huntington’s disease, Creutzfeldt-Jakob disease, Lou Gehrig’s disease, spinal cerebellar degeneration, spinal cerebellar ataxia, prion disease, cognitive dysfunction, senile dementia, Lewy body dementia, frontotemporal dementia, vascular dementia, alcoholic dementia, presenile dementia, Machado-Joseph disease, myodystonia, multiple system atrophy, progressive supranuclear palsy, Friedreich ataxia, temporal lobe epilepsy, and stroke.

In some embodiments, the reconstituted high density lipoprotein or the pharmaceutical composition is administered in an amount sufficient to inhibit aggregation of amyloid-beta (Aβ) or to maintain brain tissue homeostasis.

Hereinafter, the present disclosure will be described in more detail through the examples, but the following examples are for illustrative purposes only and are not intended to limit the scope of the present disclosure.

### EXAMPLE 1

### Preparation Method of rHDL Nanoparticles Using Microfluidic Device

### 1-1. Design of Microfluidic Device for Preparing rHDL Nanoparticles

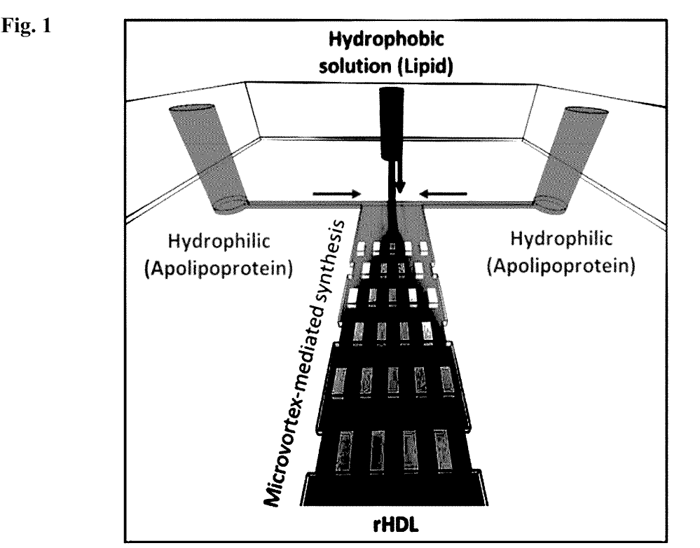

A microfluidic device for preparing reconstituted high density lipoprotein (rHDL) nanoparticles by mixing phospholipids and apolipoproteins was designed (FIG. 1). The microfluidic device comprises three inlets and one outlet, and a hydrophobic phospholipid and a hydrophobic drug were injected into an inlet located in the middle of the three inlets, and a hydrophilic apolipoprotein and a hydrophilic drug were injected into two inlets located on both sides. In addition, a micropillar structure was introduced into the microfluidic device to effectively mix lipids and apolipoproteins.

### 1-2. Preparation 1 of rHDL Nanoparticles

The rHDL nanoparticles containing a phospholipid (DMPC) and an apolipoprotein were prepared using the microfluidic device by the following method.

A DMPC (1,2-dimyristoyl-sn-glycero-3-phosphocholine) solution in absolute ethanol and an apolipoprotein solution in PBS were prepared. Thereafter, one syringe was filled with about 0.8 mL of the DMPC solution in absolute ethanol at a concentration of 0.83 mg/ml, and the other two syringes were each filled with the same amount of about 1.25 ml (a total of about 2.5 ml) of the apolipoprotein solution in PBS at a concentration of 0.2 mg/ml, and then all syringes were defoamed.

Each syringe needle and the inlet of the microfluidic device were connected using a tube, and PBS was flowed at an outlet rate of 1 mL/min to wash the microfluidic device. Thereafter, using a syringe pump, the injection flow rate of the DMPC solution was set to 0.8 mL/min, and the injection flow rate of the apolipoprotein solution was set to 2.2 mL/min. The rHDL nanoparticles prepared through the outlet of the microfluidic device were obtained, and the obtained nanoparticles were mixed with PBS, and then purified three times at 4° C. for 20 minutes at the maximum speed of a centrifuge using a 10 K filter. About 250 µL of the nanoparticle solution was allowed to be remained in the residue after the last purification and stored at 4° C.

### 1-3. Preparation 2 of rHDL Nanoparticles

The rHDL nanoparticles containing a phospholipid (DPPC) and an apolipoprotein were prepared in the same manner as in 1-2 above.

### 1-4. Preparation 3 of rHDL Nanoparticles

The rHDL nanoparticles containing a phospholipid (POPC) and an apolipoprotein were prepared in the same manner as in 1-2 above.

### 1-5. Effect of Micropillar Structure

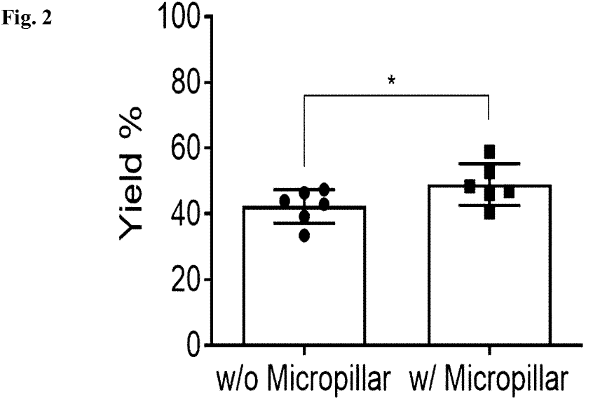

In order to confirm the mixing efficiency of lipids and apolipoproteins depending on the presence or absence of micropillar structures, the distribution of phospholipids in the microfluidic device was observed using simulation and red ink. As a result, as shown in FIG. 1, it was confirmed that the micropillar structure generated a microvortex to more efficiently mix phospholipids and apolipoproteins. In addition, as a result of confirming the production yield of rHDL nanoparticles through BCA protein quantification, as shown in FIG. 2, it was confirmed that the production yield in the presence of micropillars was enhanced compared to the case without micropillars.

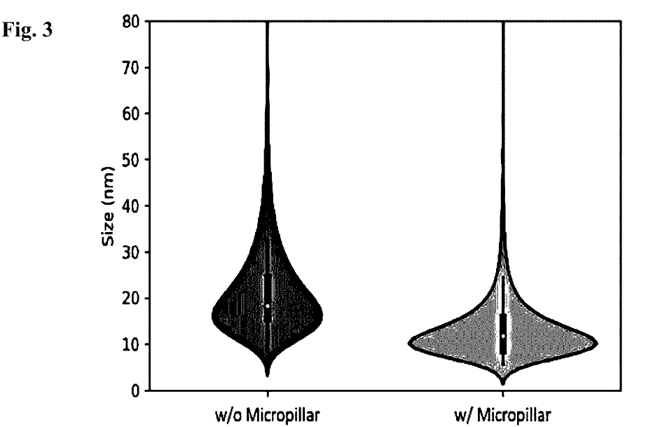

In addition, as shown in FIG. 3, as a result of measuring DLS data after dissolving the rHDL nanoparticles prepared in two types of microfluidic devices in PBS, it was confirmed that the size of the rHDL nanoparticles prepared in the microfluidic device with micropillars was much more uniform compared to the case without micropillars, and the degree of aggregation was decreased, thereby increasing the homogeneity of the particle size.

### EXAMPLE 2

### Optimization of Blending Ratio of Phospholipid and Apolipoprotein E3

In order to optimize the synthesis blending ratio of phospholipids and apolipoproteins used for preparing rHDL nanoparticles, the average size and size distribution of rHDL nanoparticles depending on the synthesis blending ratio were compared by the following method.

### 2-1. rHDL Nanoparticles Containing Phospholipid (DMPC) and Apolipoprotein

In the same manner as in Example 1-2 above, the rHDL nanoparticles prepared by varying the blending ratio of phospholipid (DMPC) and apolipoprotein were dissolved in PBS, and then the change in the particle size distribution depending on the aggregation phenomenon of nanoparticles through dynamic light scattering (DLS) was measured using Zetasizer Nano ZS. The results were plotted by intensity, volume, and number.

In order to artificially prepare stable HDL, rHDL must have a high density and small size, and stable rHDL must be prepared in large quantities to be utilized as medicines.

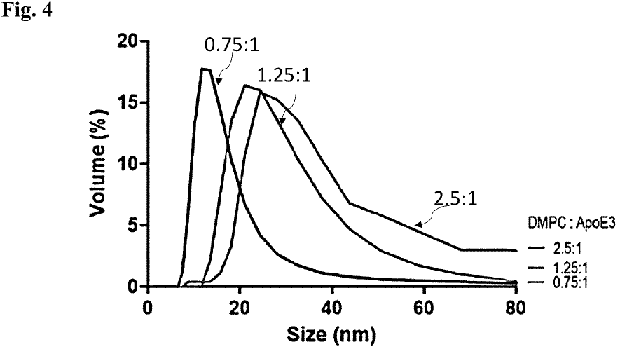

As shown in FIG. 4, the case where the synthesis blending weight ratio of phospholipid (DMPC) and apolipoprotein E3 (GenBank: ARQ79461.1) is 0.75:1 was compared with the case where it is 1.25: 1 and the case where it is 2.5:1. It was confirmed that when the synthesis blending weight ratio was 0.75:1, rHDL, a final product with a small and uniform nanoparticle size, could be obtained.

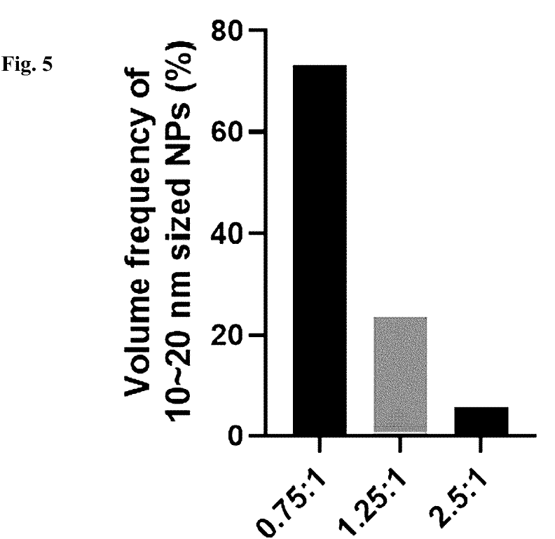

HDL naturally produced in the human body has a diameter of 20 nm or less, and it can be seen that most of the particles of the rHDL of the present disclosure have a size of 20 nm or less. Specifically, as shown in FIG. 5, it was confirmed that the distribution of HDL having a stable particle size of 10-20 nm was calculated to be 70% or more for the case where the synthesis blending weight ratio was 0.75: 1, and it was 20% or more for the case where the synthesis blending weight ratio was 1.25: 1, but it was less than 5% for the case where the synthesis blending weight ratio was 2.5:1.

It can be seen that when the synthesis blending weight ratio of phospholipid (DMPC) and apolipoprotein E3 exceeds 2.5:1, a larger amount of phospholipid is included than the amount required for the formation of nanoparticles, and thus the remaining phospholipids form a cluster by linking the stably formed rHDL with each other or further aggregate to generate a phospholipid aggregate.

In addition, it was confirmed that when the synthesis blending weight ratio of phospholipid (DMPC) and apolipoprotein E3 is less than 0.25:1, a larger amount of apolipoprotein is included than the amount required for the formation of nanoparticles, and thus apolipoprotein that is not formed into nanoparticles aggregates.

In general, since apolipoprotein is relatively expensive compared to phospholipids, it is more preferable for mass production not to include apolipoprotein in excess of a required amount during synthesis. Therefore, for efficient mass production, it is preferable to remove the phospholipids together with the organic solvent after synthesis by including relatively inexpensive phospholipids in excess of the required amount.

Therefore, after the synthesis of nanoparticles, the remaining phospholipids that are not synthesized into nanoparticles must be removed as much as possible through the process of removing the organic solvent, so that uniform and stable rHDL can be produced. For this reason, the weight ratio of phospholipid and apolipoprotein contained in the final product, rHDL nanoparticles, appears to be smaller than the synthesis blending weight ratio.

### 2-2. rHDL Nanoparticles Containing Phospholipid (DPPC) and Apolipoprotein

In the same manner as in Example 1-3 above, the rHDL nanoparticles prepared by varying the blending ratio of phospholipid (DPPC) and apolipoprotein were dissolved in PBS, and then the change in the particle size distribution depending on the aggregation phenomenon of nanoparticles through dynamic light scattering (DLS) was measured using Zetasizer Nano ZS.

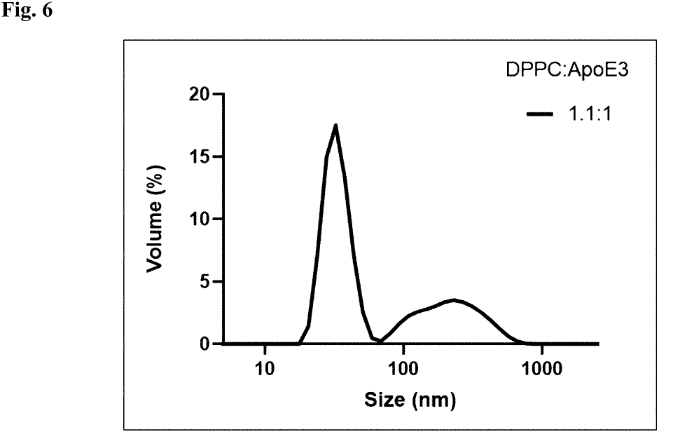

As shown in FIG. 6, it was confirmed that when the synthesis blending weight ratio of phospholipid (DPPC) and apolipoprotein E3 was 1.1:1, rHDL, a final product with a small and uniform nanoparticle size, could be obtained.

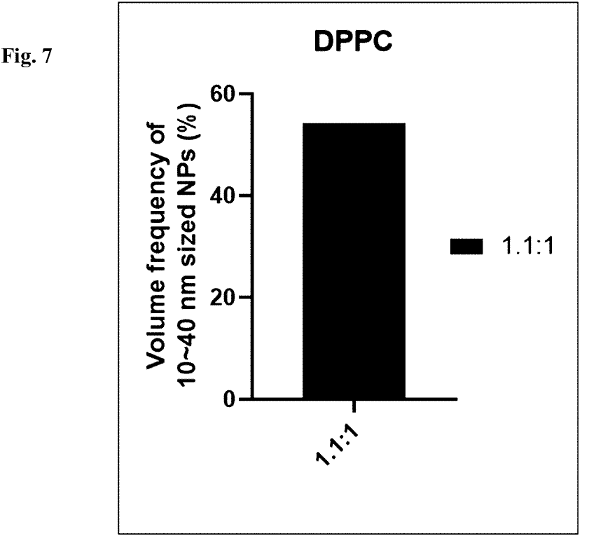

Specifically, as shown in FIG. 7, it was confirmed that the distribution of HDL having a particle size of 40 nm or less was calculated to be 50% or more for the case where the synthesis blending weight ratio was 1.1:1.

It was confirmed that when the synthesis blending weight ratio of phospholipid (DPPC) and apolipoprotein E3 exceeds 2.5:1, a larger amount of phospholipid is included than the amount required for the formation of nanoparticles, and thus the remaining phospholipids form a cluster by linking the stably formed rHDL with each other or further aggregate to generate a phospholipid aggregate.

In addition, it was confirmed that when the synthesis blending weight ratio of phospholipid (DPPC) and apolipoprotein E3 is less than 0.5:1, the size distribution of the aggregate was not uniform due to the increase in the instability of the constituent substances due to the amount of phospholipids smaller than the amount required for the formation of nanoparticles.

### 2-3. rHDL Nanoparticles Containing Phospholipid (POPC) and Apolipoprotein

In the same manner as in Example 1-4 above, the rHDL nanoparticles prepared by varying the blending ratio of phospholipid (POPC) and apolipoprotein were dissolved in PBS, and then the change in the particle size distribution depending on the aggregation phenomenon of nanoparticles through dynamic light scattering (DLS) was measured using Zetasizer Nano ZS.

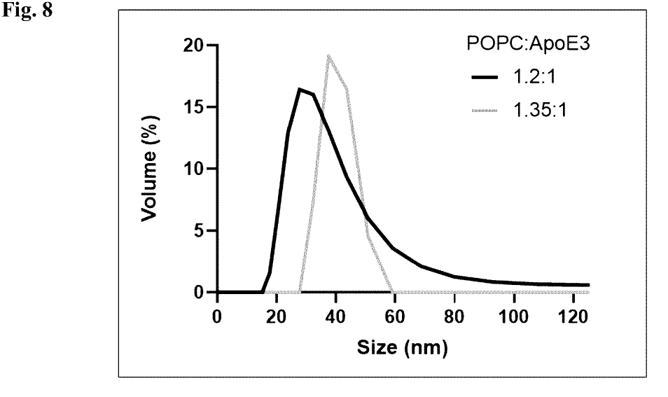

As shown in FIG. 8, the case where the synthesis blending weight ratio of phospholipid (POPC) and apolipoprotein E3 is 1.2:1 was compared with the case where it is 1.35:1. It was confirmed that when the synthesis blending weight ratio was 1.2:1, rHDL, a final product with a small and uniform nanoparticle size, could be obtained.

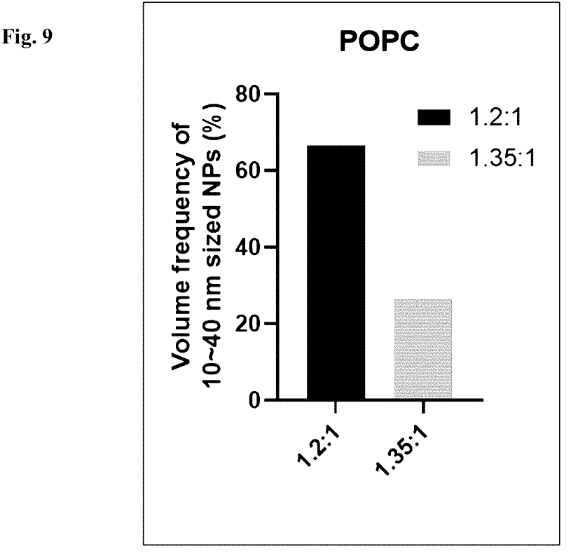

Specifically, as shown in FIG. 9, it was confirmed that the distribution of HDL having a particle size of 40 nm or less was calculated to be 60% or more for the case where the synthesis blending weight ratio was 1.2:1, and it was calculated to be 20% or more for the case where the synthesis blending weight ratio was 1.35:1.

It was confirmed that when the synthesis blending weight ratio of phospholipid (POPC) and apolipoprotein E3 exceeds 2.5:1, a larger amount of phospholipid is included than the amount required for the formation of nanoparticles, and thus the remaining phospholipids form a cluster by linking the stably formed rHDL with each other or further aggregate to generate a phospholipid aggregate.

In addition, it was confirmed that when the synthesis blending weight ratio of phospholipid (POPC) and apolipoprotein E3 is less than 0.5:1, the size distribution of the aggregate was not uniform due to the increase in the instability of the constituent substances due to the amount of phospholipids smaller than the amount required for the formation of nanoparticles.

### EXAMPLE 3

### Preparation of rHDL Nanoparticles Containing Apolipoprotein E2 or E3

The rHDL nanoparticles containing apolipoprotein E2 (GeneBank: ARQ79459) were prepared in the same manner as in Examples 1 and 2 of the preparation method of the rHDL nanoparticles containing apolipoprotein E3. Thereafter, the size distribution of the particles was measured using DLS in the same manner as in Example 2, and this was compared with the rHDL nanoparticles containing apolipoprotein E3.

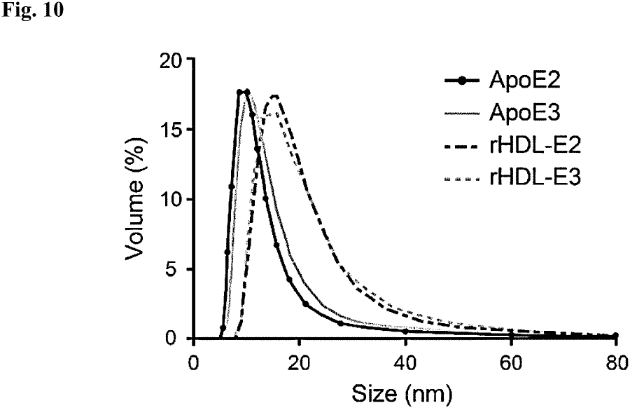

As shown in FIG. 10, there was almost no difference in shape between the rHDL nanoparticles containing apolipoprotein E3 and the rHDL nanoparticles containing apolipoprotein E2, and it was confirmed that nanoparticles could be prepared under the same conditions.

### EXAMPLE 4

### Morphology of rHDL Nanoparticles Containing Apolipoprotein E3

According to the preparation method of Example 1 above, rHDL nanoparticles with a synthesis blending weight ratio of phospholipid (DMPC) and apolipoprotein E3 of 0.75:1 were prepared, and their morphological characteristics were observed using transmission electron microscopy (TEM). The rHDL nanoparticles were dissolved in PBS, and then placed on a nickel grid, negatively stained, dried sufficiently, and then photographed.

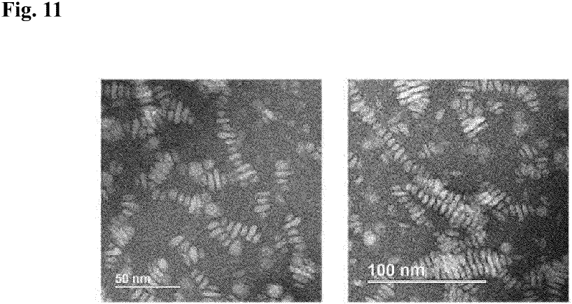

As shown in FIG. 11, it was confirmed that the rHDL nanoparticles containing apolipoprotein E3 exhibited a discoidal shape in which two layers of apolipoproteins were wrapped around phospholipids in a belt shape.

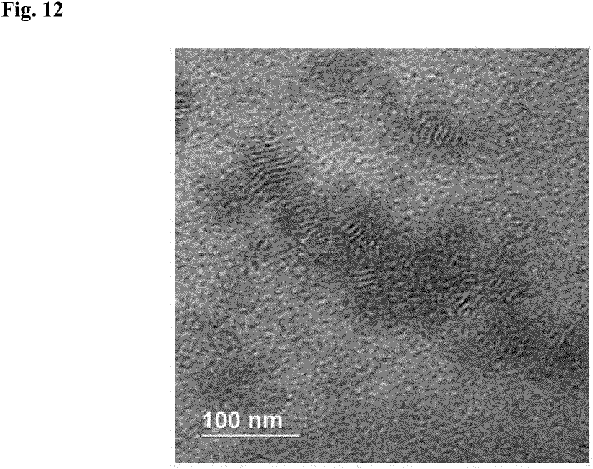

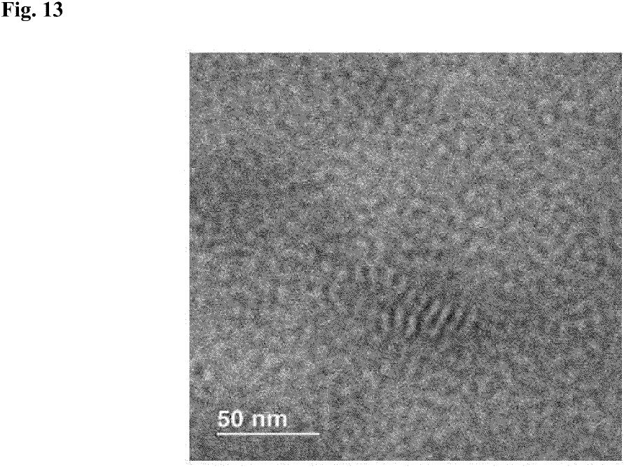

On the other hand, as shown in FIGS. 12 and 13, it can be seen that the stable rHDL with a discoidal shape is reduced when the weight ratio is 1.25: 1, and the stable rHDL is hardly observed when the weight ratio exceeds 2.5:1.

When the weight ratio exceeds 2.5:1, the remaining phospholipids connect the already generated stable rHDL like a cloud to increase the particle size of rHDL, and in this case, the rHDL has an unstable form, so it is impossible to maintain the content of rHDL at a constant concentration in the body. In addition, apolipoprotein (ApoE) passes through the BBB via the low density lipoprotein receptor (LDLR) in the body or plays a role in transporting amyloid-beta out of the brain, and phospholipids surround these apolipoproteins like a cloud, which can make it difficult for rHDL to perform its functions.

### EXAMPLE 5

### Preparation of Hybrid rHDL Nanoparticles Containing Several Types of Apolipoproteins

The rHDL nanoparticles of the present disclosure may have different functions and effects depending on the type and composition of the apolipoprotein surrounding the phospholipid. Therefore, in this example, hybrid rHDL nanoparticles composed of different types of apolipoprotein surrounding the phospholipid were prepared.

In order to prepare rHDL nanoparticles containing both apolipoprotein A1 (GenBank: AAS68227.1) and apolipoprotein E3 with excellent biocompatibility, a DMPC solution at a concentration of 1.8 mg/mL, an apolipoprotein A1 solution and an apolipoprotein E3 solution at a concentration of 0.2 mg/ml were used together, and rHDL nanoparticles were prepared according to Example 1 above.

In addition, it was confirmed that when the apolipoprotein E2 solution and the apolipoprotein E3 solution were used together, the hybrid rHDL nanoparticles containing apolipoproteins E2 and E3 could be prepared. In order to prepare the hybrid nanoparticles of the present disclosure, 1.25 mL of a solution containing ApoE2 and ApoE3, respectively, at a concentration of 0.1 mg/mL was prepared and mixed, and 0.8 mL of a DMPC solution at a concentration of 0.83 mg/mL was prepared, and the rHDL nanoparticles were prepared according to Example 1. As a result, as shown in FIG. 10, it was confirmed that the hybrid rHDL nanoparticles containing apolipoproteins E2 and E3 could be prepared as uniform nanomaterials.

When the apolipoprotein A1 solution and the apolipoprotein E3 solution are used together, the rHDL nanoparticles containing apolipoprotein A1, the rHDL nanoparticles containing apolipoprotein E3, and the hybrid rHDL nanoparticles containing apolipoproteins A1 and E3 can be all present immediately after preparation. As a result of confirming through ELISA, it was confirmed that the hybrid rHDL nanoparticles were generated in a ratio of about 10% of the total nanoparticles.

Eventually, in the conventional incubation method, an apolipoprotein is mixed with a phospholipid carrier and left. Since the apolipoprotein is agglomerated with each other, even if apolipoproteins E2 and E3 are added to the culture dish simultaneously, the E2 and E3 proteins spontaneously fuse with each other, and thus the hybrid rHDL is not generated. However, it can be seen that hybrid rHDL nanoparticles are generated through the preparation method of the present disclosure.

### EXAMPLE 6

### Composition of Phospholipid and Apolipoprotein in rHDL Nanoparticles Containing apolipoprotein E3

In order to confirm the final composition ratio of the phospholipid and apolipoprotein of the rHDL nanoparticles prepared according to Examples 1 and 2 above, BCA assay and lipid quantification kit were used for quantification. The results are shown in Table 1 below.

TABLE 1

| No. | Protein (mg/mL) | Phospholipid (mg/mL) | Phospholipid:Protein |
|---|---|---|---|
| 1 | 0.77 | 0.26 | 0.34:1 |
| 2 | 1.24 | 0.42 | 0.34:1 |
| 3 | 1.11 | 0.51 | 0.47:1 |
| 4 | 1.19 | 0.46 | 0.39:1 |
| 5 | 2.42 | 0.76 | 0.31:1 |
| 6 | 2.33 | 0.67 | 0.29:1 |

As shown in Table 1 above, it was confirmed that the phospholipid:apolipoprotein ratio of a total of 6 groups of rHDL nanoparticles prepared at different times was 0.2:1 to 0.5:1. That is, it can be seen that the phospholipid:protein after the synthesis of rHDL prepared in a synthesis blending weight ratio of 0.75:1 of phospholipid:apolipoprotein is 0.2:1 to 0.5:1. Therefore, it can be seen that the phospholipid content in the final product, rHDL, is reduced as excess phospholipids are removed together with the organic solvent during the production process of rHDL.

The rHDL of the present disclosure contains phospholipids in the two-layered apolipoprotein structure, so it was confirmed that the phospholipid:apolipoprotein after the synthesis of rHDL prepared at the optimal synthesis blending ratio for mass production was 0.2:1 to 0.5:1.

However, when excess phospholipids remain without being removed together with the organic solvent during the production process of rHDL, the phospholipid:apolipoprotein ratio after the synthesis of rHDL can be 0.2:1 to 2.5:1, or when excess phospholipids remains without being completely removed together with the organic solvent, the ratio can be 0.2: 1 to 1.5:1.

### EXAMPLE 7

### Measurement and Calculation of Density of rHDL Nanoparticles

The density of rHDL nanoparticles was calculated based on the molecular weight of apolipoprotein E3 used for synthesis, the length of materials used for synthesis, and the volume range of the final synthesized product through the DLS measurement results.

The mass information of rHDL nanoparticles is as follows. The molecular weight of apolipoprotein E3 is 35.20 kDa, and apolipoprotein E3 forms two layers in the final synthesized product, so the total mass of the constituent protein is calculated to be 70.40 kDa. In addition, the preferred final lipid:apolipoprotein ratio of the rHDL nanoparticles prepared according to Examples 1 and 2 above was confirmed to be 0.2:1 to 0.5:1, and thus the mass of the lipid contained in the final synthesized product was calculated to be 14.08 to 35.20 kDa. Therefore, the molecular weight of the final rHDL nanoparticles was calculated to be a value of 84.48 to 105.60 kDa (140.24X 10⁻²¹ to 175.30X 10⁻²¹ g), which is the sum of the constituent proteins and lipids.

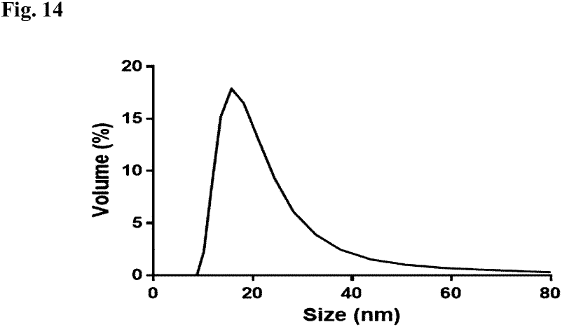

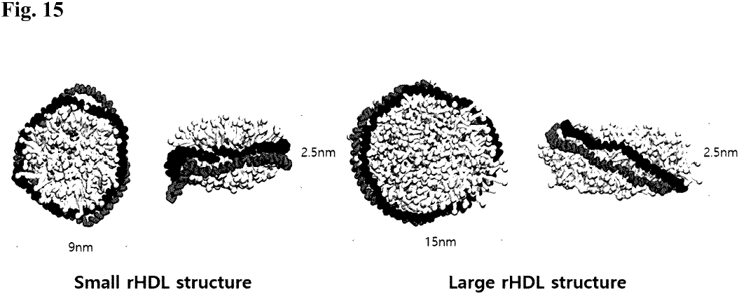

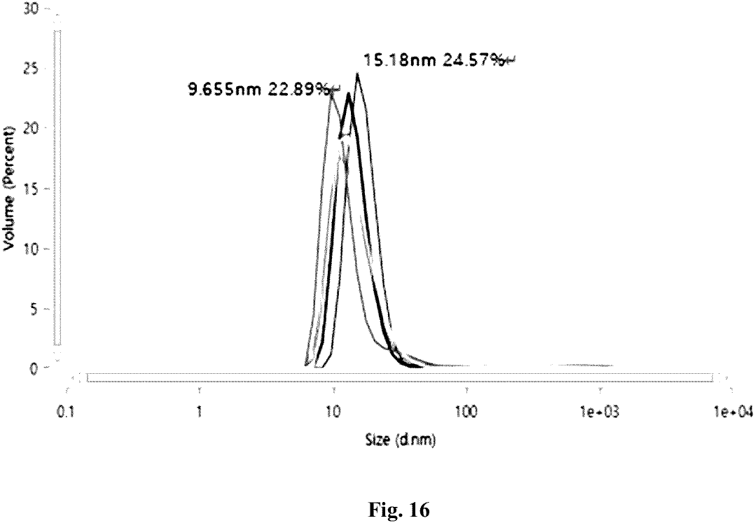

The volume information of rHDL nanoparticles is as follows. The morphology of the rHDL nanoparticles was inferred through simulation, and the results are shown in FIGS. 14 to 16. As shown in FIGS. 14 to 16, it was confirmed that the long axis (diameter) length of the rHDL nanoparticles containing apolipoprotein E3 had a distribution of 8 to 15 nm. Assuming that the short axis (height) of the rHDL nanoparticles having a discoidal shape was 2.5 nm, the volume of a sphere or ellipsoid was calculated to be 125.6 to 441.6 nm³.

Therefore, the density of rHDL nanoparticles was calculated as a density distribution of 0.3 to 1.2 g/mL through the mass and volume estimated through the experimental data.

### TEST EXAMPLE 1

### Confirmation of Aβ Aggregation Inhibitory Effect of rHDL Nanoparticles

In order to confirm the amyloid-beta (Aβ) aggregation inhibitory effect of the rHDL nanoparticles prepared in the above example, the following experiment was carried out.

1 µM A(β1-42 labeled with 488 fluorescence and the rHDL nanoparticles at three concentrations (0.01, 0.1, 0.5 µM) were prepared, and a 96-well plate was simultaneously treated therewith, and then the fluorescence values after 0, 10, 20, 30, 40, 50, 60, 120, 150, 240, 300, 360, 720, and 1440 minutes were photographed, respectively. For comparison, the fluorescence value of the plate treated with 488-Aβ alone was also checked. When 488-Aβ is aggregated, it is self-quenched and the fluorescence value is lowered. Therefore, if the fluorescence value when treated with rHDL nanoparticles is higher than when treated with 488-Aβ alone, it can be interpreted that it has an effect of inhibiting Aβ aggregation.

### 1-1. rHDL Nanoparticles Containing Apolipoprotein E2 or E3

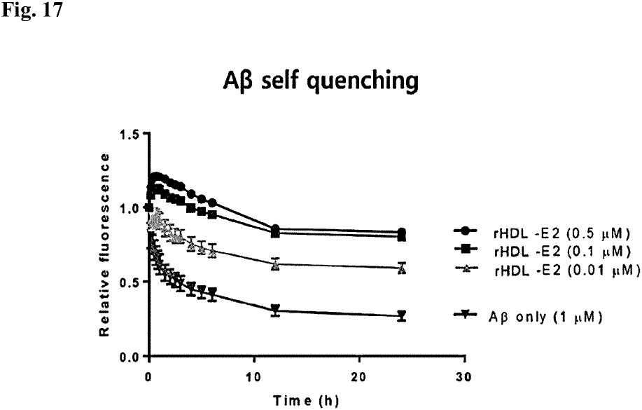

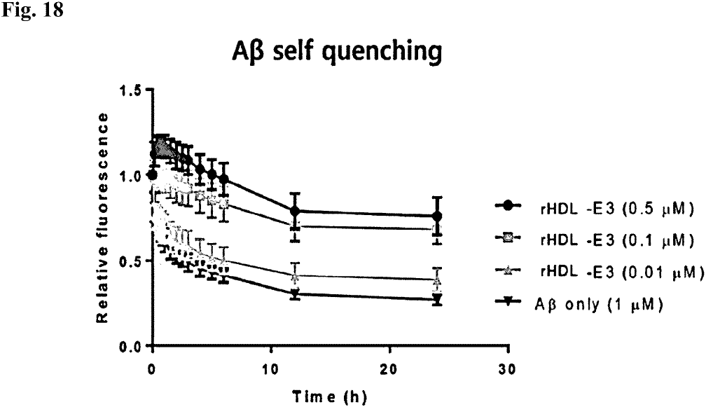

As shown in FIGS. 17 and 18, it was confirmed that when treated with 488-Aβ alone, 488-Aβ was aggregated and self-quenched over time, and the fluorescence value was lowered. It was confirmed that the fluorescence value when treated with the rHDL nanoparticles containing apolipoprotein E2 or E3 was higher than when treated with 488-Aβ alone. In addition, it was confirmed that the fluorescence value appeared higher as the concentration of the rHDL nanoparticles was increased.

Therefore, it can be seen that the rHDL nanoparticles of the present disclosure have an excellent Aβ aggregation inhibitory effect.

### 1-2. Hybrid rHDL Nanoparticles Containing Apolipoproteins E2 and E3 simultaneously

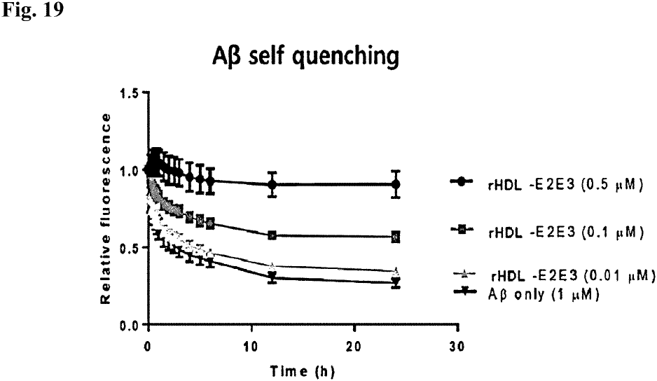

As shown in FIG. 19, it was confirmed that the fluorescence value when treated with the hybrid rHDL nanoparticles containing apolipoproteins E2 and E3 was higher than when treated with 488-Aβ alone. As the concentration of the hybrid rHDL nanoparticles was increased, the fluorescence value was measured to be higher, and in particular, when treated with 0.5 µM of the hybrid rHDL nanoparticles, the fluorescence value was hardly decreased. Therefore, it was confirmed that it has a significant effect of inhibiting Aβ aggregation.

### TEST EXAMPLE 2

### Comparison of Aβ Aggregation Inhibitory Effect Depending on Type of Apolipoprotein Contained in rHDL Nanoparticles

In order to compare the effect difference depending on the type of apolipoprotein contained in the rHDL nanoparticles, amyloid-beta (Aβ) aggregation inhibitory effect of the rHDL nanoparticles containing apolipoprotein E2 or E3 at the same concentration of 0.5 µM was compared with amyloid-beta (Aβ) aggregation inhibitory effect of the hybrid rHDL nanoparticles containing apolipoproteins E2 and E3 in the same manner as in Test Example 1.

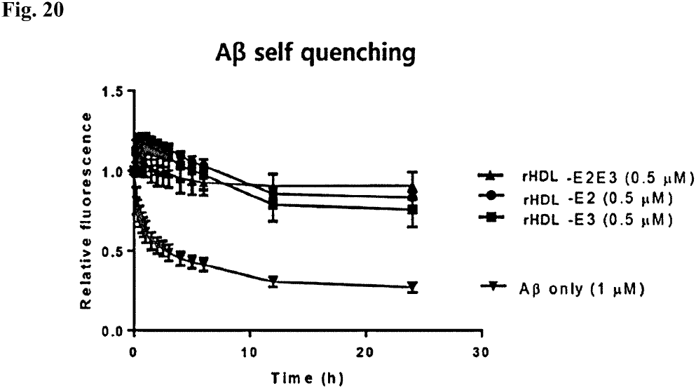

As shown in FIG. 20, it was confirmed that both the rHDL nanoparticles containing apolipoprotein E2 or E3 and the hybrid rHDL nanoparticles containing apolipoproteins E2 and E3 exhibited an excellent Aβ aggregation inhibitory effect, and in particular, the hybrid rHDL nanoparticles exhibited the highest effect after 10 hours. Within the first 20 minutes, a phenomenon, in which the aggregated Aβ was deaggregated before the start of the experiment and the fluorescence value was temporarily increased, was observed.

### TEST EXAMPLE 3

### Comparison of Aβ Aggregation Inhibitory Effect Between Pure Apolipoprotein and rHDL Nanoparticles

In order to confirm whether Aβ aggregation inhibitory effect is exhibited only by treatment with apolipoprotein alone, not the rHDL nanoparticles of the present disclosure, it was treated with Aβ and apolipoprotein E3, or it was treated with Aβ and the rHDL nanoparticles containing apolipoprotein E3 at 1:0.1, and the change in fluorescence value was confirmed in the same manner as in Test Example 1.

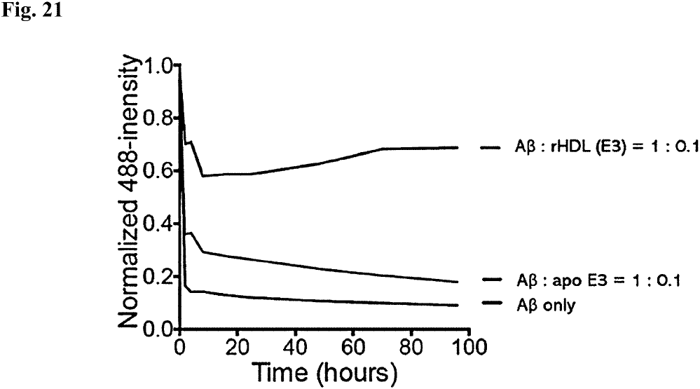

As shown in FIG. 21, even when it was treated with apolipoprotein E3, the fluorescence value was higher than when it was treated with Aβ alone, but the fluorescence value was significantly higher when it was treated with the rHDL nanoparticles containing apolipoprotein E3. Therefore, it was confirmed that the Aβ aggregation inhibitory effect of the rHDL nanoparticles of the present disclosure was very excellent.

### TEST EXAMPLE 4

### Comparison of Transported Amount of rHDL Nanoparticles in Endothelial Cells of Blood-brain Barrier

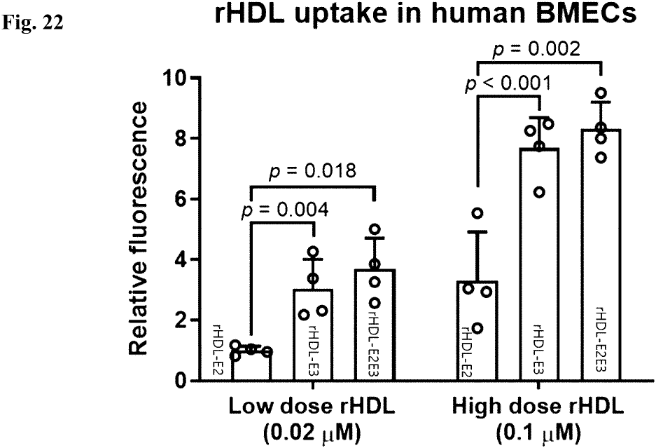

An experiment on the degree of transport (transcytosis) into brain tissue mediated by brain microvascular endothelial cells (BMEC) for the rHDL nanoparticles containing apolipoprotein E2 or E3, and the hybrid rHDL nanoparticles containing apolipoproteins E2 and E3 was conducted, and the results are shown in FIG. 22.

As shown in FIG. 22, it was confirmed that in the case of the hybrid rHDL nanoparticles containing apolipoproteins E2 and E3, transport (transcytosis) into brain tissue mediated by brain microvascular endothelial cells (BMEC) of blood-brain barrier was excellent compared to the rHDL nanoproteins containing single apolipoprotein E2 or E3. This means that when the same amount of nanomaterial is supplied to the receptors of brain microvascular endothelial cells to which E2 and E3 respond, respectively, the hybrid rHDL can be more effectively absorbed into cells than the single apolipoprotein (E2 or E3) rHDL.

Therefore, it can be seen that when rHDL is used as a therapeutic agent or delivery agent in the future, it can pass through the brain microvascular endothelial cell barrier more efficiently even with a minimum dose, thereby enabling more effective drug expression and drug delivery.

### TEST EXAMPLE 5

### Comparison of Transported Amount of rHDL Nanoparticles in Astrocytes of Blood-brain Barrier

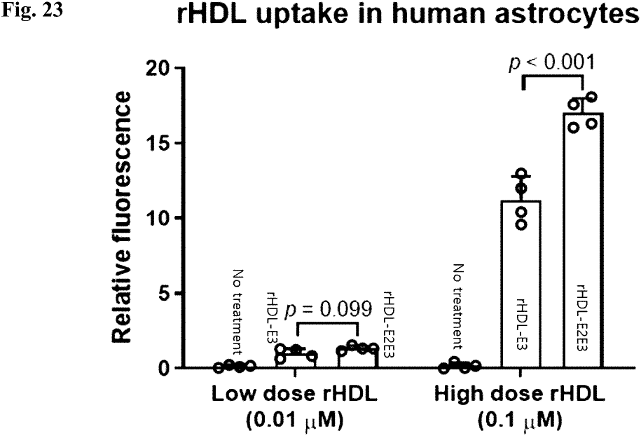

An experiment on the degree of transport mediated by astrocytes of the blood-brain barrier for the rHDL nanoparticles containing apolipoprotein E2 or E3, and the hybrid rHDL nanoparticles containing apolipoproteins E2 and E3 was conducted, and the results are shown in FIG. 23.

As shown in FIG. 23, in the case of the hybrid rHDL nanoparticles containing apolipoproteins E2 and E3, transport mediated by astrocytes of the blood-brain barrier was excellent compared to the single apolipoprotein (E2 or E3) rHDL. Thus, it can be seen that it exhibits a more excellent effect on maintaining homeostasis in the brain tissue.

This means that when the same amount of nanomaterial is supplied to the receptors of astrocytes to which E2 and E3 respond, respectively, the hybrid rHDL nanoparticles containing apolipoproteins E2 and E3 can respond to the receptors to the maximum. Therefore, it can be seen that the maximum effect can be exhibited with the minimum dose.

### TEST EXAMPLE 6

### Comparison of Removal of Brain Tissue Amyloid Beta in Animal Model of Alzheimer’s Disease

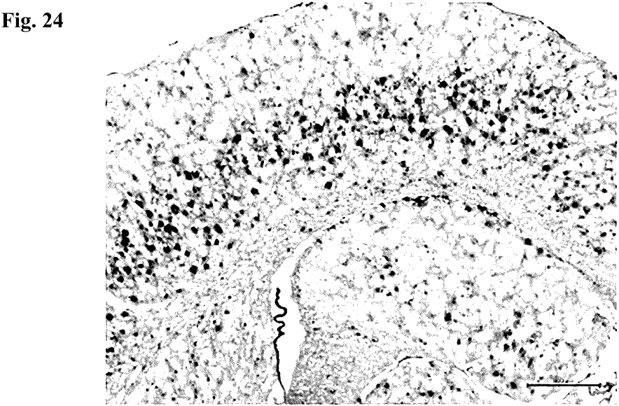

An experiment on the removal of brain tissue amyloid beta in an animal model of Alzheimer’s disease for the rHDL nanoparticles containing apolipoprotein E3 was conducted, and the results are shown in FIGS. 24 to 27.

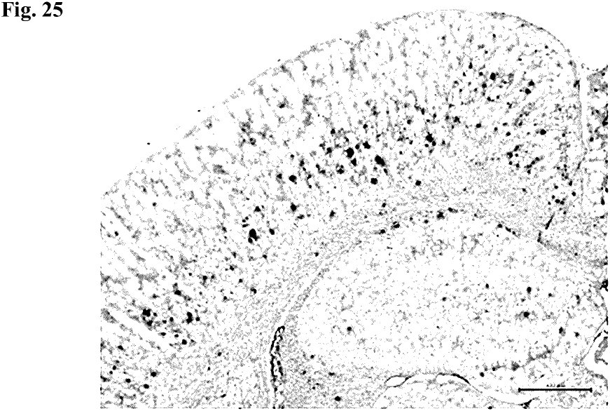

The same number of female and male normal mice and an animal model of Alzheimer’s disease (5xFAD) were divided into a control group and a group administered with rHDL nanoparticles, respectively, and saline was administered to the control group by i.v. injection, and the rHDL nanoparticles of the present disclosure containing apolipoprotein E3 (2.5 mg/kg) were administered to the group administered with rHDL nanoparticles once every 3 days for a total of 33 times. 24 hours after the last administration, the brain was extracted, and the left brain was fixed with a 4% PFA (paraformaldehyde) solution, and the fixed brain tissue was frozen through an OCT compound and cut using a microtome to have a thickness of 4 µm. The cut tissue section was attached to a slide and reacted with Aβ-specific antibody using a DAB (3,3′-diaminobenzidine tetrahydrochloride salt) coloring agent. The stained slides were imaged using a confocal microscope, and the results are shown in FIGS. 24 and 25.

As shown in FIG. 24, in an animal model of Alzheimer’s disease (5xFAD) administered with saline for 3 months, excessive Aβ deposition was observed in the brain. However, as shown in FIG. 25, it can be seen that the Aβ deposition is significantly reduced when the rHDL nanoparticles containing apolipoprotein E3 are administered. It can be seen that the rHDL nanoparticles containing E3 can effectively remove Aβ protein in brain tissue that causes Alzheimer’s disease.

In addition, in order to compare the Aβ concentration in the cerebrospinal fluid of each experimental group, after anesthetizing the mice before brain extraction of the mice, the cerebrospinal fluid (CSF) was collected through the cisterna magna between the cerebellum and the medulla oblongata. In order to compare the Aβ concentration in the plasma, the cerebrospinal fluid was obtained, and then blood was collected through the jugular vein to obtain about 0.13 mL of whole blood in a tube treated with heparin (5 IU/mL). The collected blood was centrifuged to separate plasma.

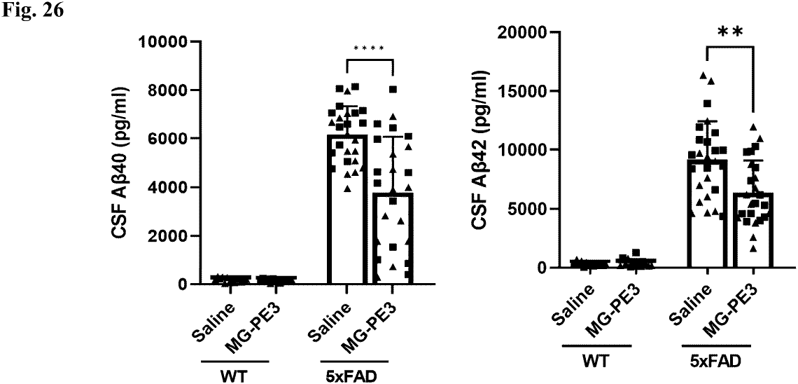

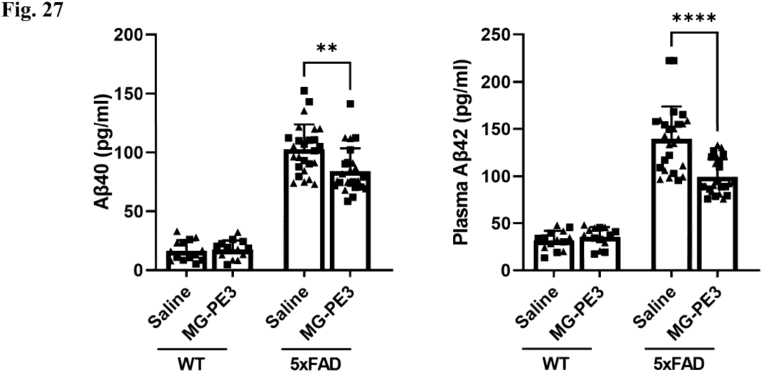

As a result, as shown in FIGS. 26 and 27, it was confirmed that the Aβ concentration in the cerebrospinal fluid and plasma in an animal model of Alzheimer’s disease (5xFAD) administered with the rHDL nanoparticles containing apolipoprotein E3 was significantly reduced compared to the group administered with saline.

1. A reconstituted high density lipoprotein (rHDL) comprising a phospholipid; and apolipoprotein E.

2. The reconstituted high density lipoprotein (rHDL) according to claim 1, wherein the reconstituted high density lipoprotein (rHDL) has been generated from a mixture comprising the phospholipid, and apolipoprotein E at a weight ratio of 0.25: 1 to 2.5:1.

3. The reconstituted high density lipoprotein (rHDL) according to claim 1, wherein the reconstituted high density lipoprotein (rHDL) has been generated from a mixture comprising the phospholipid, and apolipoprotein E at a weight ratio of 0.5:1 to 1.5:1.

4. The reconstituted high density lipoprotein (rHDL) according to claim 1, wherein the weight ratio of the phospholipid, and apolipoprotein E in the reconstituted high density lipoprotein (rHDL) is 0.2:1 to 2.5:1.

5. The reconstituted high density lipoprotein (rHDL) according to claim 4, wherein the weight ratio of the phospholipid, and apolipoprotein E in the reconstituted high density lipoprotein (rHDL) is 0.2:1 to 1.5:1.

6. The reconstituted high density lipoprotein (rHDL) according to claim 5, wherein the weight ratio of the phospholipid, and apolipoprotein E in the reconstituted high density lipoprotein (rHDL) is 0.2:1 to 0.5:1.

7. The reconstituted high density lipoprotein (rHDL) according to claim 1, wherein the density of the reconstituted high density lipoprotein (rHDL) is 0.1 to 2.0 g/ml.

8. The reconstituted high density lipoprotein (rHDL) according to claim 7, wherein the density of the reconstituted high density lipoprotein (rHDL) is 0.2 to 1.5 g/ml.

9. The reconstituted high density lipoprotein (rHDL) according to claim 8, wherein the density of the reconstituted high density lipoprotein (rHDL) is 0.3 to 1.2 g/ml.

10. The reconstituted high density lipoprotein (rHDL) according to claim 1, comprising apolipoprotein E2, E3, or both E2 and E3.

11. The reconstituted high density lipoprotein (rHDL) according to claim 1, further comprising apolipoprotein A1.

12. The reconstituted high density lipoprotein (rHDL) according to claim 10, wherein the reconstituted high density lipoprotein (rHDL) is free of apolipoprotein other than apolipoprotein E2, E3 or A1.

13. The reconstituted high density lipoprotein (rHDL) according to claim 10, wherein the reconstituted high density lipoprotein (rHDL) is free of apolipoprotein E4.

14. The reconstituted high density lipoprotein (rHDL) according to claim 1, wherein the phospholipid is at least one selected from the group consisting of 1,2-dioleoyl-sn-glycero-3-phosphatidylcholine (DOPC), egg phosphatidylcholine (EPC), dilauroylphosphatidylcholine (DLPC), 1,2-dimyristoyl-sn-glycero-3-phosphocholine (DMPC), dipalmitoylphosphatidylcholine (DPPC), distearoylphosphatidylcholine (DSPC), 1-myristoyl-2-palmitoylphosphatidylcholine (MPPC), 1-palmitoyl-2-myristoylphosphatidylcholine (PMPC), 1-palmitoyl-2-stearoylphosphatidylcholine (PSPC), 1-stearoyl-2-palmitoyl phosphatidylcholine (SPPC), 1,2-distearoyl-sn-glycero-3-phosphocholine (DAPC), 1,2-diarachidoyl-sn-glycero-3-phosphocholine (DBPC), 1,2-dieicosanoyl-sn-glycero-3-phosphocholine (DEPC), palmitoyloleoylphosphatidylcholine (POPC), lysophosphatidylcholine, dilinoleoylphosphatidylcholine, distearoylphosphatidylethanolamine (DSPE), dimyristoylphosphatidylethanolamine (DMPE), dipalmitoylphosphatidylethanolamine (DPPE), palmitoyloleoylphosphatidylethanolamine (POPE), lysophosphatidylethanolamine, N1-[2-((1S)-1-[(3-aminopropyl)amino]-4-[di(3-amino-propyl)amino]butylcarboxamido)ethyl]-3,4-di[oleyloxy]-benzamide) (VL-5), dioctadecylamidoglycylspermine 4-trifluoroacetic acid (DOGS), 3β-[N-(N′,N′-dimethylaminoethane)-carbamoyl]cholesterol (DC-Cho1), 1,2-di-O-octadecenyl-3-trimethylammonium propane (DOTMA), 1,2-dioleyl-3-trimethylammonium-propane (DOTAP), (1,2-dioleyloxypropyl)-3-dimethylhydroxyethyl ammonium bromide (DORIE), 1,2-dimyristyloxy-propyl-3-dimethyl-hydroxy ethyl ammonium bromide (DMRIE), 2,3-dioleyloxy-N-[2-(sperminecarboxamido)ethyl]-N,N-dimethyl-1-propanaminium trifluoroacetate (DOSPA), N-(3-aminopropyl)-N,N-dimethyl-2,3-bis(dodecyloxy)-1-propanammonium bromide (GAP-DLRIE), N-t-butyl-N′-tetradecyl-3-tetradecylaminopropionamidine (diC14-amidine), ethylphosphocholine (ethyl PC), dimethyldioctadecylammonium bromide (DDAB), N4-cholesteryl-spermine (GL67), 1,2-dioleyloxy-3-dimethylaminopropane (DODMA), and D-Lin-MC3-DMA (MC3, DLin-MC3-DMA), DLin-KC2-DMA, and DLin-DMA.

15. A pharmaceutical composition comprising the reconstituted high density lipoprotein according to claim 1 and a pharmaceutically acceptable excipient.

16. The pharmaceutical composition according to claim 15, comprising a first set of the reconstituted high density lipoprotein comprising apolipoprotein E2 and a second set of the reconstituted high density lipoprotein comprising apolipoprotein E3.

17. The pharmaceutical composition according to claim 15, comprising the reconstituted high density lipoprotein comprising both apolipoprotein E2 and apolipoprotein E3.

18-20. (canceled)

21. A method for preparing a reconstituted high density lipoprotein (rHDL) comprising a phospholipid; and apolipoprotein E, comprising the steps of:
injecting a first hydrophilic solution comprising a first apolipoprotein into a first inlet of a microfluidic device comprising three inlets and one outlet,
injecting a phospholipid solution into a second inlet of the microfluidic device, and
injecting a second hydrophilic solution comprising a second apolipoprotein into a third inlet of the microfluidic device.

22. The method for preparing a reconstituted high density lipoprotein (rHDL) according to claim 21, wherein the microfluidic device comprises the second inlet in the middle and the first and the third inlet on both sides of the microfluidic device.

23. (canceled)

24. The method for preparing a reconstituted high density lipoprotein (rHDL) according to claim 21, wherein both the first apolipoprotein and the second apolipoprotein are apolipoprotein E3.

25. (canceled)

26. (canceled)

27. The method for preparing a reconstituted high density lipoprotein (rHDL) according to claim 21, wherein the first hydrophilic solution, the phospholipid solution, and the second hydrophilic solution are injected concurrently.

28. The method for preparing a reconstituted high density lipoprotein (rHDL) according to of claim 21, further comprising collecting the reconstituted high density lipoprotein (rHDL) from the outlet.

29. The method for preparing a reconstituted high density lipoprotein (rHDL) according to of claim 21, wherein the weight ratio of the phospholipid, and apolipoprotein E injected into the inlets of the microfluidic device is 0.25:1 to 2.5:1.

30. The method for preparing a reconstituted high density lipoprotein (rHDL) according to claim 29, wherein the weight ratio of the phospholipid, and apolipoprotein E injected into the inlets of the microfluidic device is 0.5:1 to 1.5:1.

31. A reconstituted high density lipoprotein (rHDL) prepared by the preparation method according to claim 21.

32. A method for preventing or treating a neurodegenerative disease in a subject, comprising administering to the subject the reconstituted high density lipoprotein according to claim 1.

33. The method for preventing or treating a neurodegenerative disease according to claim 32, wherein the neurodegenerative disease is selected from the group consisting of Parkinson’s disease, Alzheimer’s disease, Pick’s disease, Huntington’s disease, Creutzfeldt-Jakob disease, Lou Gehrig’s disease, spinal cerebellar degeneration, spinal cerebellar ataxia, prion disease, cognitive dysfunction, senile dementia, Lewy body dementia, frontotemporal dementia, vascular dementia, alcoholic dementia, presenile dementia, Machado-Joseph disease, myodystonia, multiple system atrophy, progressive supranuclear palsy, Friedreich ataxia, temporal lobe epilepsy, and stroke.

34. (canceled)
